(19) United States
(12) Patent Application Publication
(10) Pub. No.: US 2016/0058279 A1
(43) Pub. Date: Mar. 3, 2016

(54) SYSTEM AND METHOD FOR IMAGING AN EYE FOR DIAGNOSIS

(71) Applicant: DRESSCOM, INC., Coto Laurel, PR (US)

(72) Inventor: Miguel Torres, Juana Diaz, PR (US)

(21) Appl. No.: 14/838,492

(22) Filed: Aug. 28, 2015

Related U.S. Application Data
(60) Provisional application No. 62/044,053, filed on Aug. 29, 2014.

(51) Int. Cl. A61B 3/00; A61B 3/14

(52) U.S. Cl. CPC A61B 3/0025; A61B 3/14; A61B 3/0058

(57) ABSTRACT

A mobile device includes a programmable processor operatively coupled to a memory, a display subsystem, and a camera subsystem and is configured to execute non-transitory computer-executable code, which instructs the programmable processor to: determine whether a camera image input received from the camera subsystem includes a representation of an iris from a human eye; capture a candidate image from the camera image input, the candidate image being including the representation of the iris; analyze the representation of the iris to determine whether the candidate image is suitable for processing as a diagnostic image based upon the representation of the iris meeting predetermined criteria; perform a diagnostic analysis on the representation of the iris in the diagnostic image in response to determining that the candidate image is suitable for further processing as the diagnostic image, the diagnostic analysis arriving at diagnostic results; and display the diagnostic results using the display subsystem.

### CROSS REFERENCE TO RELATED APPLICATIONS

Priority is claimed to U.S. Provisional Application No. 62/044,053, filed Aug. 29, 2015, the disclosure of which is incorporated herein by reference in its entirety.

### FIELD OF THE INVENTION

The field of the present invention relates to systems and methods for the imaging of an eye for diagnostic purposes, particularly systems and methods that may be operated by an individual using a portable electronic device to obtain a preliminary diagnosis from an image of their own eye.

### BACKGROUND OF THE INVENTION

Blood tests in labs are presently the only methods by which a person may tested and receive professional diagnosis for certain types of health-related markers. These markers may include as cholesterol levels, blood glucose levels, blood hemoglobin levels, and arterial blood pressure. In well-populated or well-developed regions of the world, access to labs and doctors to receive blood testing and professional diagnosis is generally not an issue (although lab analyses may be overused), but in less populated or less developed regions, access to labs for blood analysis is quite often an issue.

However, even well-populated or well-developed regions, people may have issues with going to a lab for blood work. These issues may include: a fear of blood, needles, and/or pain; being highly susceptible to illness due to exposure to pathogens in the clinical setting; the inability to afford the cost of blood work; and the inability to obtain transportation to the lab, especially in the case of being handicapped. (These same issues may also apply in less populated or less developed regions.)

Regardless of the reason why an individual cannot get blood work done, many would benefit from a quick and easy way to either obtain a preliminary diagnosis for certain health markers and/or a way to obtain a professional diagnosis without having to visit a lab for blood work.

### SUMMARY OF THE INVENTION

The present invention is directed toward a system and method for capturing an image of an eye for purposes of receiving a diagnosis. In certain embodiments of the invention, the system and method enable an individual to capture a diagnostic image of the eye using a handheld device in order to obtain a preliminary diagnosis based on an analysis of the iris in the diagnostic image. In certain other embodiments of the invention, the diagnostic image of the eye may be forwarded to a physician for review and a professional diagnosis.

In a first separate aspect of the present invention, a mobile device includes: a memory; a display subsystem; a camera subsystem; and a programmable processor operatively coupled to the memory, the display subsystem, and the camera subsystem and configured to execute non-transitory computer-executable code. The code instructs the programmable processor to: determine whether a camera image input received from the camera subsystem includes a representation of an iris from a human eye; capture a candidate image from the camera image input in response to determining that the camera image input includes the representation of the iris; analyze the representation of the iris in the candidate image to determine whether the candidate image is suitable for further processing as a diagnostic image based upon the representation of the iris in the candidate image meeting predetermined criteria; perform a diagnostic analysis on the representation of the iris in the diagnostic image in response to determining that the candidate image is suitable for further processing as the diagnostic image, the diagnostic analysis arriving at diagnostic results; and display the diagnostic results using the display subsystem.

In a second separate aspect of the present invention, a method of imaging an eye for performing a diagnosis includes: determining, by a programmable processor, whether a camera image input received from a camera subsystem includes a representation of an iris from a human eye; capturing, by the programmable processor, a candidate image from the camera image input in response to determining that the camera image input includes the representation of the iris; analyzing, by the programmable processor, the representation of the iris in the candidate image to determine whether the candidate image is suitable for further processing as a diagnostic image based upon the representation of the iris in the candidate image meeting predetermined criteria; performing, by the programmable processor, a diagnostic analysis on the representation of the iris in the diagnostic image in response to determining that the candidate image is suitable for further processing as the diagnostic image, the diagnostic analysis arriving at diagnostic results; and displaying, by the programmable processor using the display subsystem, the diagnostic results.

In a third separate aspect of the present invention, a mobile device includes: a memory; a display subsystem; a camera subsystem; and a programmable processor operatively coupled to the memory, the display subsystem, and the camera subsystem and configured to execute non-transitory computer-executable code. The code instructs the programmable processor to: determine whether a camera image input received from the camera subsystem includes a representation of an iris from a human eye; display the camera image input using the display subsystem until the candidate image is captured, wherein iris image alignment guidelines include an alignment circle displayed as an overlay on the displayed camera image input; display a time out message using the display subsystem in response to being unable to determine, after a predetermined period of time, that the camera image input includes the representation of the iris; capture a candidate image from the camera image input in response to determining that the camera image input includes the representation of the iris, the candidate image having a predetermined number of pixels; display the candidate image in response to the candidate image being captured; analyze the representation of the iris in the candidate image, in response to the candidate image being captured, to determine whether the candidate image is suitable for further processing as a diagnostic image based upon the representation of the iris in the candidate image meeting predetermined criteria; perform a diagnostic analysis on the representation of the iris in the diagnostic image in response to determining that the candidate image is suitable for further processing as the diagnostic image, the diagnostic analysis arriving at diagnostic results; and display the diagnostic results using the display subsystem in response to the diagnostic analysis being performed.

Accordingly, an improved system and method for imaging an eye for diagnosis are disclosed. Advantages of the improvements will be apparent from the drawings and the description herein.

### BRIEF DESCRIPTION OF THE DRAWINGS

The foregoing summary, as well as the following detailed description of the exemplary embodiments, will be better understood when read in conjunction with the appended drawings. It should be understood, however, that the invention is not limited to the precise arrangements and instrumentalities shown in the following figures:

FIG. 1 is a schematic view of a device for imaging an eye for diagnosis;

FIG. 2 is a flow chart showing a process for imaging an eye for diagnosis; and

FIGS. 3A-H are screenshots from a mobile programmable device showing one implementation of a process for imaging an eye for diagnosis.

### DETAILED DESCRIPTION OF THE INVENTION

Features of the present invention may be implemented in software, hardware, firmware, or combinations thereof. The programming processes described herein are not limited to any particular embodiment, and may be implemented in an operating system, application program, foreground or background processes, driver, or any combination thereof. The programming processes may be compiled as executable code to be executed on a single programmable processor or multiple programmable processors.

The programmable processors described herein may be any central processing unit (CPU), microprocessor, micro-controller, computational, or programmable device or circuit configured for executing programming processes (e.g., executable code). Various processors may be embodied in any suitable type of hardware and may include all the usual ancillary components necessary to form a functional data processing and communication device including without limitation a bus, software and data storage such as volatile and non-volatile memory, input/output devices, graphical user interfaces (GUIs), removable data storage, and wired and/or wireless communication interface devices including Wi-Fi, Bluetooth, LAN, without limitation, unless expressly recited in the claims.

Computer-executable instructions or programs (e.g. software or code) and data described herein may be programmed into and tangibly embodied in a non-transitory computer-readable medium that is accessible to and retrievable by a respective processor as described herein which configures and directs the processor to perform the desired functions and processes by executing the instructions encoded in the medium. A device embodying a programmable processor configured to such non-transitory computer-executable instructions or programs is referred to hereinafter as a “programmable device,” or for certain embodiments, a “mobile device,” or just a “device” for short. It should be noted that non-transitory “computer-readable medium” as described herein may include, without limitation, any suitable volatile or non-volatile memory including random access memory (RAM) and various types thereof, read-only memory (ROM) and various types thereof, USB flash memory, and magnetic or optical data storage devices (e.g. internal/external hard disks, floppy discs, magnetic tape CD-ROM, DVD-ROM, optical disk, ZIP™ drive, Blu-ray disk, and others), which may be written to and/or read by a processor operably connected to the medium.

In certain embodiments, the present invention may be embodied in the form of computer-implemented processes and apparatuses such as processor-based data processing and communication systems or computer systems for practicing those processes. The present invention may also be embodied in the form of software or computer program code embodied in a non-transitory computer-readable storage medium, which when loaded into and executed by the data processing and communications systems or computer systems, the computer program code segments configure the processor to create specific logic circuits configured for implementing the processes.

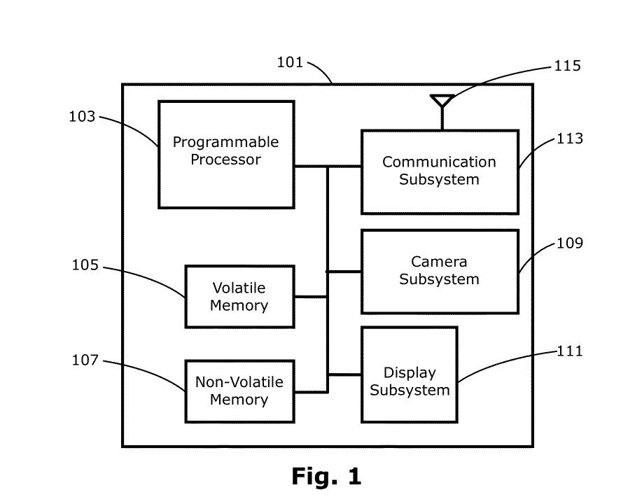

An exemplary mobile device 101 is shown in FIG. 1. The mobile device 101 includes a programmable processor 103, a volatile memory 105, a non-volatile memory 107, a camera subsystem 109, a display subsystem 111, and a communication subsystem 113. Other features common to mobile programmable devices may also be included with the mobile device 101, such as a power switch, buttons and/or switches to indicate settings and/or interact with the software, an audio speaker, and audio out. The non-volatile memory 107 is used by the programmable processor 103 to store executable code for instructing the programmable processor 103 to perform the functions described herein. The non-volatile memory 107 may also be used by the programmable processor 103 to store operational settings, user information, data, and any other information that may be desirable to store long term for use with the mobile device 101. The volatile memory 105 may be used by the programmable processor 103 to store transitory data, e.g., data that is needed only at the time of a particular use and that may be erased by the programmable processor 103 without impairing functionality of the mobile device 101.

The camera subsystem 109 includes one or more sets of camera lenses (not shown) and at least one charge coupled device (CCD) image sensor (not shown) operatively coupled together to form an electronic imaging system (i.e., a camera). The camera subsystem 109 generates camera image input, which is a digital representation of objects imaged by the camera lenses onto the CCD image sensor. The camera input image may be in the form of a series of still images, or alternatively, it may be in the form of a video. The programmable processor 103 receives the camera input image and is programmed to capture an image from it. For purposes of the process described herein, the programmable processor 103 should be able to capture and image having at least 5 megapixels from the camera image input received from the camera subsystem 109. In certain embodiments, the mobile device 101 may generate captured images having less than 5 megapixels, however, a reduction in the imaging capability of the mobile device 101 may hinder the diagnosis capabilities described herein.

The display subsystem 111 includes a display screen (not shown), which may be an LCD screen, an OLED screen, or any other type of display screen. In certain embodiments, the display subsystem 111 may include a touch sensitive display which allows the user to interact with the software through the display subsystem 111 itself. In certain embodiments, the display screen of the display subsystem 111 and the entrance aperture of the camera subsystem 109 are placed on the same side of the mobile device 101. This placement makes it easier for a single user to image a body part, such as an eye, using the camera subsystem 109 while at the same time viewing the image on the display screen of the display subsystem 111.

The communication subsystem 113 is operationally coupled to one or more appropriate antennas 115 and includes a radio frequency (RF) transceiver configured to wireless sly transmit and receive modulated RF signals at one or more radio frequencies according to one or more pre-established communication protocols, such as Wi-Fi, Bluetooth, cellular mobile communications, or any other type of wireless communication protocol. The communication subsystem 113 is therefore configured for wireless communications with other devices. The communication subsystem 113 may also be configured to communicate over a wired network with other devices by inclusion of one or more wired communication ports, such as USB, USB2, Ethernet, or any other type of wired communication technology.

In the following description, the imaging of a body part for purposes of obtaining either of a provisional or professional diagnosis is described within the context of imaging an eye. Many of the features and functionality discussed, however, will have wider applicability to providing diagnoses, whether such diagnoses are provisional or professional, based on imaging other parts of the body that may be readily imaged by a portable device without being invasive of the body.

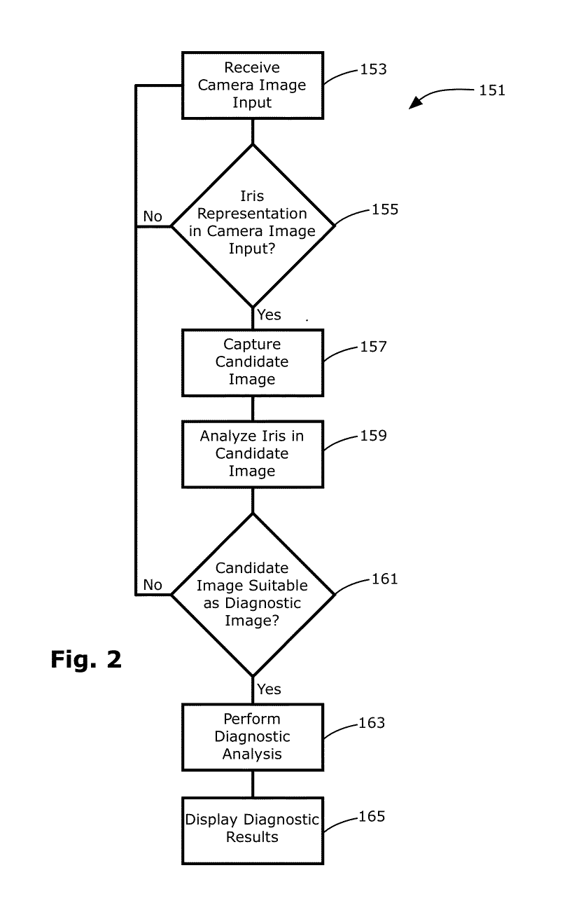

The process of imaging a human eye using the mobile device 101 is shown in the flowchart 151 of FIG. 2. As a preliminary step of the process, the programming of the mobile device is activated by the user, and upon activation, the programming may instruct the programmable processor to display a splash screen to the user. The splash screen may display information that is desirable to convey, such as the type of analyses that will be performed following acquisition of a suitable image of the human eye (or of another body part which is intended as the subject of the image acquisition and subsequent diagnostic analysis).

From the splash screen, the user may activate the image acquisition and diagnosis process, which begins by the camera subsystem generating the camera image input that is received 153 by the programmable processor. Upon activation by the user, the programmable processor begins analyzing the camera image input to determine 155 whether the camera image input includes a representation of an iris from a human eye. This is a coarse recognition step of the imaging process to ensure that the desired body part that is to be the subject of analysis later on in the process, i.e., the human eye, and in particular the iris of the human eye, is actually being imaged by the camera subsystem.

The process determining 155 whether the camera image input includes a representation of an iris from a human eye may be accomplished by mathematical analysis of images from the camera image data. For example, the upper and lower eyelids of the human eye, when the eye is open, can be approximated by parabolic curves, such that the curves of each of the upper and lower eyelids in an image may be identified by an approximate fit with a parabolic curve. The curvature of one or both the iris and the pupil may similarly be used to determine whether the camera image input includes a representation of an iris from a human eye. Other factors, such as the coloration of the pupil may be used to determine whether the camera image input includes a representation of an iris from a human eye. Any other appropriate feature of an eye, alone or in combination with other identifiable features, may be used to determine whether the camera image input includes a representation of an iris from a human eye.

While the programmable processor receives and analyzes the camera image input from the camera subsystem for the presence of a representation of an iris, images from the camera image input are displayed to the user on the display screen of the display subsystem. Display of the camera image input continues until a candidate image is captured 157. By displaying images from the camera image input, the user is better able to position the mobile device, relative to the body, so that a their own eye may be imaged by the camera subsystem. A written message may also be displayed along with the images from the camera image input to inform the user about the status of the image acquisition process.

To further aid the user in positioning the mobile device, the programmable processor may display iris alignment guidelines on the display screen of the display subsystem as an overlay on the displayed images from the camera image input. The iris alignment guidelines may be in the form of the outline of a circle to show the user where to place the mobile device with respect to their eye when trying to obtain an image of the eye. The outline of the circle enables the user to position their eye so that the circular shape of the iris has approximately the same center point as the circle. In addition, the mobile device may be moved closer or further away from the user's eye so that the circular shape of the iris approximately fills the circle. The outline of the circle is present merely to provide an approximation for the position and alignment of the user's eye with respect to the mobile device in order to obtain a suitable image, as a suitable image may still be obtained by an eye positioned off center from the outline of the circle and/or an iris that does not fill or fills more than the entire outline of the circle.

The iris alignment guidelines may also include an image display window, which is shown on the display screen of the display subsystem as a window that is smaller than the entire display screen. The programmable processor may display a static image outside of the image display window, so that the user is better able to focus on getting their eye within the image display window, thereby facilitating obtaining a suitable image. The image display window may include a predetermined number of pixels, with the predetermined number of pixels corresponding to the needs of the system to capture a suitable image of the eye for further processing, and particularly, to capture a suitable image of the iris for further processing. In certain embodiments, the predetermined number of pixels for the image display window is based on obtaining a predetermined number of pixels for the iris in the resulting images of the eye. In certain embodiments, the predetermined number of pixels for the iris in the captured images is at least 400 pixels. With the predetermined number of pixels set for the iris in the captured images, the predetermined number of pixels for the image display window may be determined for a particular display screen. As will be recognized, the size of the image display window will be dependent upon the imaging capacity of the camera subsystem and the pixel density of the screen display for the display subsystem. In certain embodiments, the size of the image display window may be adjusted to better accommodate the technical specifications of the hardware.

After a predetermined period of time of trying to determine whether the camera image input includes a representation of an iris, and not being able to identify a representation of an iris within the camera image input, the programmable processor will use the display subsystem to display a time out message to the user. This time out message may be displayed to the user by displaying the splash screen with additional information about the time out.

Once the programmable processor has determined 155 that the camera image input includes a representation of an iris, then it captures 157 a candidate image from the camera input image, such that the representation of the iris is depicted in the candidate image. In response to the candidate image being captured 157 from the camera image input, the programmable processor displays the captured image on the display screen of the display subsystem. A written message may be displayed along with the captured candidate image to inform the user of the status of the process.

After capture 157 of the candidate image, the representation of the iris in the candidate image is analyzed 159 to determine 161 whether the representation of the iris is suitable for further processing as a diagnostic image. This is a fine recognition step of the imaging process to verify that the representation of the iris from a human eye in the candidate image actually includes sufficient detail to enable the analyses to be performed in subsequent steps.

In general, the representation of the iris in the candidate image meets predetermined criteria which indicate that the representation of the iris includes sufficient image detail to perform one or more desired diagnosis analyses. The predetermined criteria may include one or more of pixel density of the representation of the iris, the completeness of the representation of the iris visible in the candidate image, the coloration of the image, and the like. The pixel density of a circle within the candidate image which outlines the iris is preferably at least 400 pixels. The coloration of the candidate image may be examined to ensure that the color is not overly saturated or muted. The completeness of the representation of the iris visible in the candidate image is important to ensure that the portions of the iris that the process needs to examine are actually present in the candidate image.

In the event that the representation of the iris in the candidate image is determined not to be suitable for further processing the programmable processor returns to the step of receiving camera image input to continue attempting to capture an appropriate candidate image of the eye.

Once the candidate image is determined 161 as being suitable for use as the diagnostic image, then the programmable processor proceeds to perform a diagnostic analysis 163 on the representation of the iris in the diagnostic image. For the diagnostic analysis of the iris, known techniques of iridology may be applied. For example, the representation of the iris in the diagnostic image may be analyzed using known iris maps developed by Dr. Bernand Jensen. In embodiments using such iris maps, iris coloration, spots within the iris, and/or zones of the iris may be analyzed using known iridology techniques to arrive at a preliminary diagnosis relating to one or more of cholesterol levels, blood glucose levels, blood hemoglobin levels, and arterial blood pressure for the user. In certain embodiments, the iris in the diagnostic image may be analyzed using other criteria. Once the diagnostic analysis 163 has been performed, the programmable processor displays 165 the preliminary diagnosis on the display screen of the display subsystem.

The preliminary diagnosis arrived at by the diagnostic analysis 163 of the iris and subsequently displayed 165 is intended for the informational use of the user, and not intended as a formal diagnosis such as might be provided by a trained medical professional. Instead, the preliminary diagnosis is intended to help the user decide whether they should seek professional medical care.

In addition to displaying 165 the preliminary diagnosis, the programmable processor may also save the diagnostic image and the preliminary diagnosis to memory for later access by the user. In certain embodiments, the programmable processor may also collect identifying information about the user and store that identifying information in memory, associating it with the diagnostic image and the preliminary diagnosis. In certain embodiments, the user may also supply an email address so that the data stored in memory may be transmitted as part of an electronic message to an email recipient. The recipient of the electronic message may be the user themselves, a family member, and/or medical personnel, such as a doctor or nurse. In certain embodiments, the data stored in memory may be transmitted in an electronic message through other protocols, such as through text messages between mobile devices or through the use of any other messaging platform.

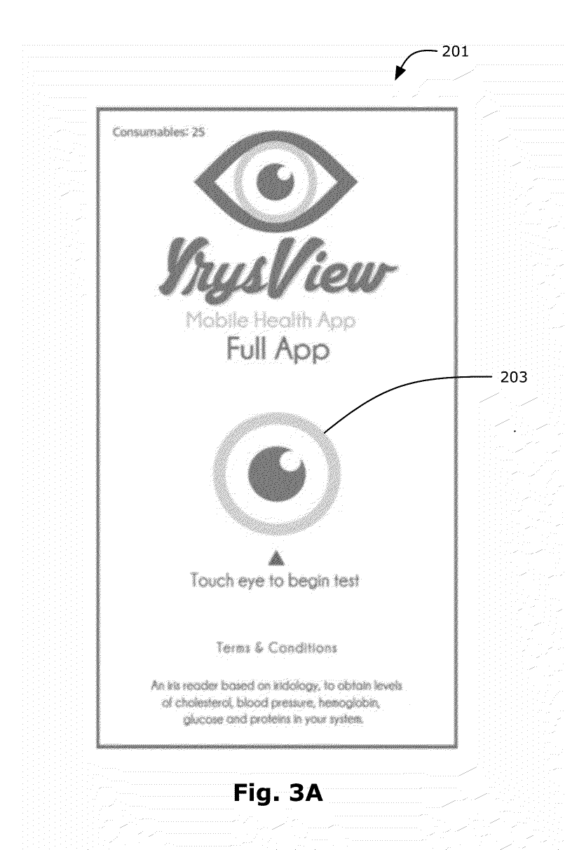

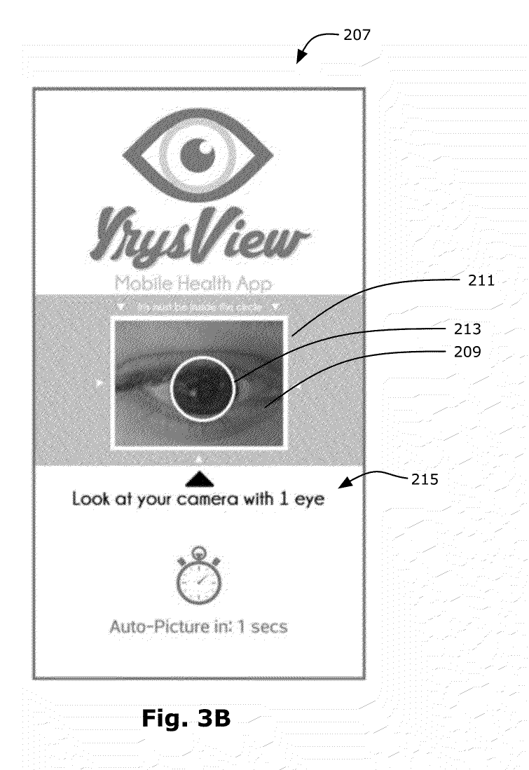

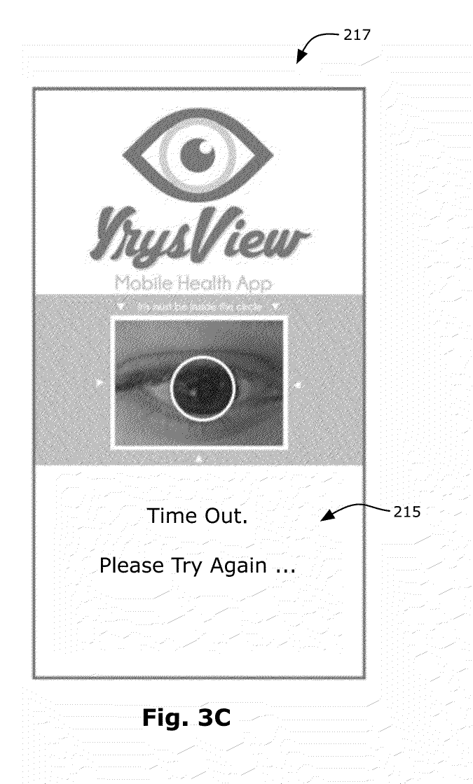

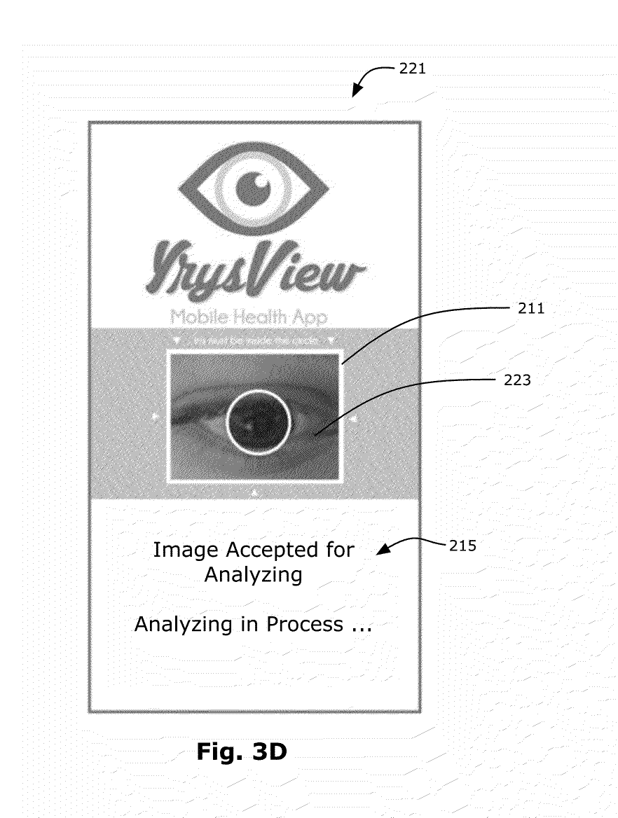

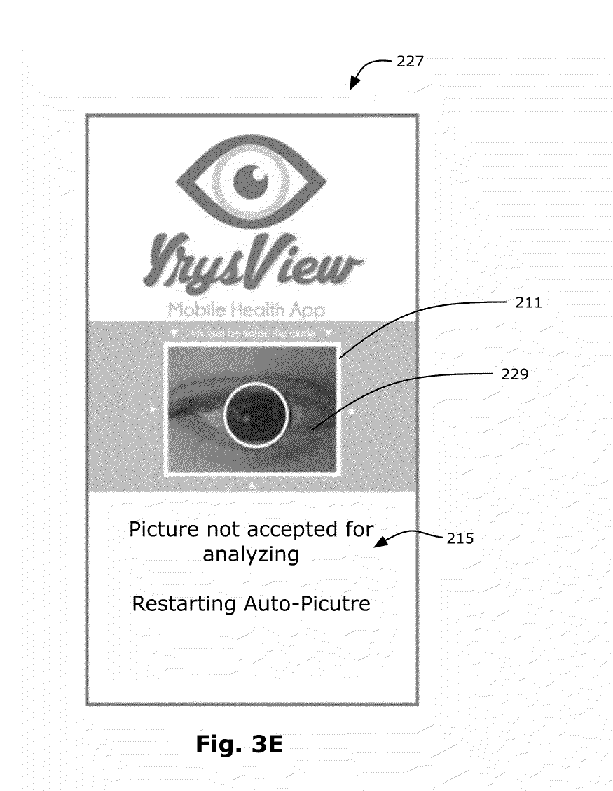

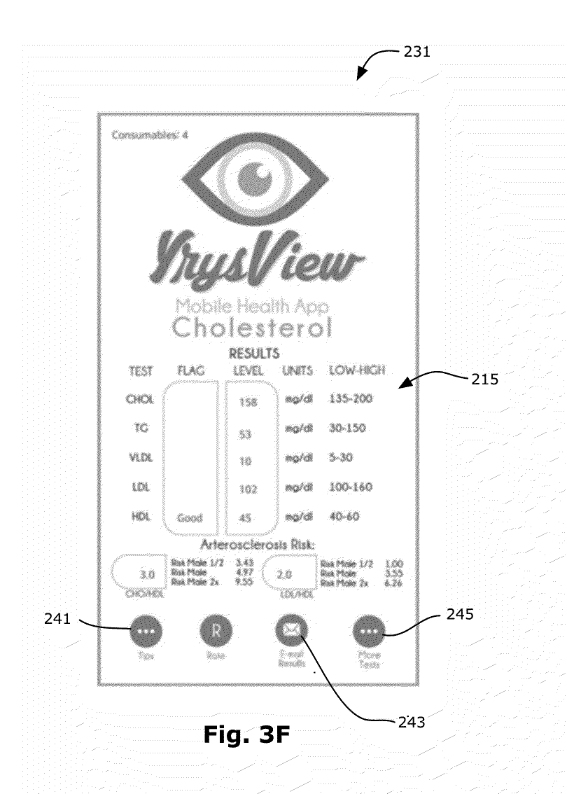

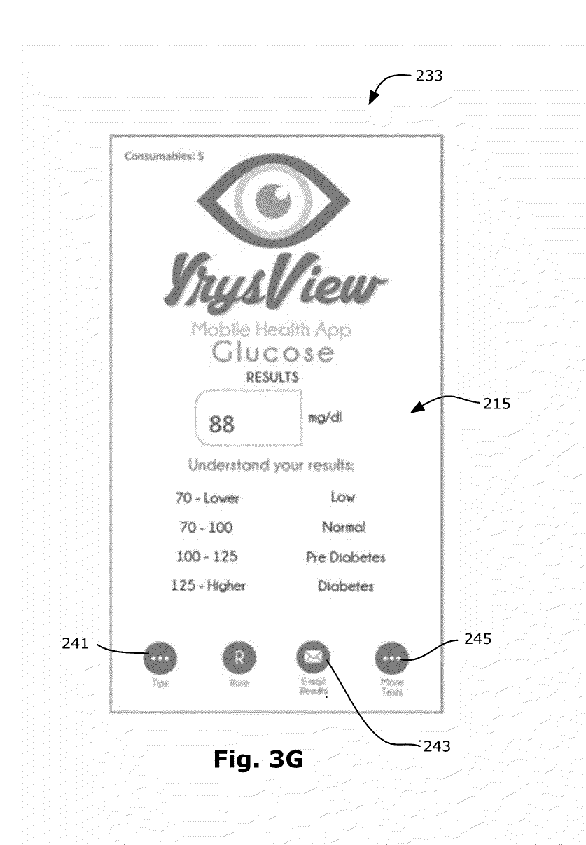

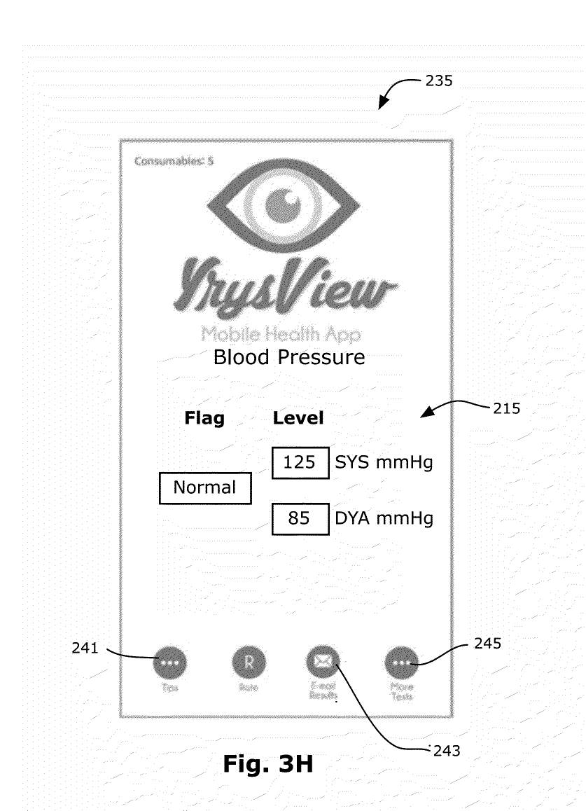

FIGS. 3A-H show screenshots from an exemplary mobile device on which the process shown in the flowchart of 151 FIG. 2 has been implemented as a programmed application (referred to as an “app”). A splash screen 201 is shown in FIG. 3A. On this splash screen 201, the user is welcomed to the app and instructed to touch the depiction of the eye 203 in the center of the screen to begin the image acquisition and diagnostic processes. When the user touches the depiction of the eye 203, the image acquisition screen 207 of FIG. 3B displayed. On this image acquisition screen 207, an image 209 from the camera image input is displayed in the image display window 211. An iris alignment circle 213 is displayed as an overlay over the image 209 within the image display window 211. The iris alignment circle 213 enables the user to position the mobile device with respect to one of their eyes so that their iris in the displayed image 209 is approximately concentric with the iris alignment circle 213. A message area 215 includes text which reports the status of the image acquisition process to the user. As discussed above, the displayed image 209 changes as the programmable processor continues to receive the camera image input as the process of identifying and capturing an appropriate image of the eye for further analysis.

In the event that the captured candidate image is not suitable for further processing as a diagnostic image, then the reset screen 217 of FIG. 3C is displayed. On this reset screen 217, the message area 215 includes text which informs the user that identification of the eye in the camera image input was not successful. Following display of the reset screen 217, the process returns to displaying the splash screen 201 of FIG. 3A.

When a candidate image is captured, the acceptance screen 221 of FIG. 3D is displayed. The acceptance screen 221 shows the candidate image 223 within the image display window 211 and the message area 215 includes text which indicates to the user that the candidate image and/or the diagnostic image is being analyzed. In the event that the candidate image is not suitable for further processing, the process reset screen 227 of FIG. 3E is displayed. The process reset screen 227 shows an image 229 from the camera image input within the image display window 211 and the message area 215 includes text which indicates to the user that a suitable image was not captured, so that the process is being reset to the image acquisition stage. The process reset screen 227 may be displayed briefly before the mobile device returns to displaying the image acquisition screen 207 of FIG. 3B.

Following successful identification and diagnostic analysis of a suitable diagnostic image, a result screen is displayed to the user. Each result screen may include a preliminary diagnosis as determined through the diagnostic analysis. A cholesterol result screen 231 is shown in FIG. 3F; a glucose result screen 233 is shown in FIG. 3G; and a blood pressure result screen 235 is shown in FIG. 3H. Each of the result screens 231, 233, 235 shows a preliminary diagnosis area 239 which includes information, in the form of health markers, relating to the health of the user, as determined through the diagnostic analysis. Each of the result screens 231, 233, 235 also include buttons for the user view further information or perform further actions. A tips button 241 allows the user to get more information about the presented information forming the basis of the preliminary diagnosis. An email button 243 allows the user to enter an email address so that the information forming the basis of the preliminary diagnosis, including the diagnostic image, may be sent through electronic messaging to a medical professional or any other designated person. A tests button 245 allows the user to access additional health tests available through the app.

A mobile device programmed to function as described above can have many benefits to improving the overall health of a user by providing a preliminary diagnosis based on images of the eye, or in alternative implementations, of other parts of the body. In addition to providing the preliminary diagnosis, the image on which the preliminary diagnosis is based may be forwarded to medical personnel to obtain a professional diagnosis. The diagnoses are able to analyze health markers such as cholesterol levels, blood glucose levels, blood hemoglobin levels, and arterial blood pressure, and these health markers can relate to illnesses such as kidney renal failure, heart attacks, leukemia, and liver failure, among others. A user obtaining a preliminary diagnosis can be better informed about their own health risks and take appropriate preventative steps for certain types of medical conditions, including (but not limited to):
Heart attack, through a preliminary diagnosis concerning levels of triglycerides, LDL cholesterol and HDL cholesterol and/or a preliminary diagnosis concerning arterial blood pressure;
Hypertension, through a preliminary diagnosis concerning arterial blood pressure;
Kidney diseases which may lead to renal failure, through a preliminary diagnosis concerning certain blood protein levels;
Diabetes, through a preliminary diagnosis concerning blood glucose levels;
Leukemia and other related diseases, through a preliminary diagnosis concerning blood hemoglobin levels; and
Auto accidents and other alcohol related health issues, through a preliminary diagnosis concerning blood alcohol levels.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

What is claimed is:

1. A mobile device comprising:
a memory;
a display subsystem;
a camera subsystem; and
a programmable processor operatively coupled to the memory, the display subsystem, and the camera subsystem and configured to execute non-transitory computer-executable code, the code instructing the programmable processor to:
determine whether a camera image input received from the camera subsystem includes a representation of an iris from a human eye;
capture a candidate image from the camera image input in response to determining that the camera image input includes the representation of the iris;
analyze the representation of the iris in the candidate image to determine whether the candidate image is suitable for further processing as a diagnostic image based upon the representation of the iris in the candidate image meeting predetermined criteria;
perform a diagnostic analysis on the representation of the iris in the diagnostic image in response to determining that the candidate image is suitable for further processing as the diagnostic image, the diagnostic analysis arriving at diagnostic results; and
display the diagnostic results using the display subsystem.

2. The mobile device of claim 1, wherein the code instructs the programmable processor to display the camera image input using the display subsystem until the candidate image is captured, and then to display the captured candidate image.

3. The mobile device of claim 2, wherein the code instructs the programmable processor to display iris image alignment guidelines using the display subsystem as an overlay on the displayed camera image input.

4. The mobile device of claim 3, wherein the iris image alignment guidelines include an iris alignment circle.

5. The mobile device of claim 3, wherein the iris image alignment guidelines include an image display window having a predetermined number of pixels.

6. The mobile device of claim 1, wherein the code instructs the programmable processor to display a time out message using the display subsystem in response to being unable to determine, after a predetermined period of time, that the camera image input includes the representation of the iris.

7. The mobile device of claim 1, wherein in response to determining that the candidate image is not suitable for further processing as the diagnostic image, the code instructs the programmable processor to return to determining whether the camera image input received from the camera subsystem includes the representation of the iris from the human eye.

8. The mobile device of claim 1, wherein the code instructs the programmable processor to store the diagnostic image and the diagnostic results to memory.

9. The mobile device of claim 8, wherein the code instructs the programmable processor to collect user identification information.

10. The mobile device of claim 9, wherein the code instructs the programmable processor to send the saved diagnostic image, the saved diagnostic results, and the user identification information to a designated recipient as part of an electronic message.

11. A method of imaging an eye for performing a diagnosis, the method comprising:
determining, by a programmable processor, whether a camera image input received from a camera subsystem includes a representation of an iris from a human eye;
capturing, by the programmable processor, a candidate image from the camera image input in response to determining that the camera image input includes the representation of the iris;
analyzing, by the programmable processor, the representation of the iris in the candidate image to determine whether the candidate image is suitable for further processing as a diagnostic image based upon the representation of the iris in the candidate image meeting predetermined criteria;
performing, by the programmable processor, a diagnostic analysis on the representation of the iris in the diagnostic image in response to determining that the candidate image is suitable for further processing as the diagnostic image, the diagnostic analysis arriving at diagnostic results; and
displaying, by the programmable processor using the display subsystem, the diagnostic results.

12. The method of claim 11, further comprising displaying, by the programmable processor using the display subsystem, the camera image input until the candidate image is captured, and then displaying the captured candidate image.

13. The method of claim 12, wherein displaying the camera image input includes displaying, by the programmable processor using the display subsystem, iris image alignment guidelines as an overlay on the displayed camera image input.

14. The method of claim 13, wherein the iris image alignment guidelines include an iris alignment circle.

15. The method of claim 13, wherein the iris image alignment guidelines include an image display window having a predetermined number of pixels.

16. The method of claim 11, further comprising displaying, by the programmable processor using the display subsystem, a time out message in response to being unable to determine, after a predetermined period of time, that the camera image input includes the representation of the iris.

17. The method of claim 11, wherein in response to determining that the candidate image is not suitable for further processing as the diagnostic image, returning to determining whether the camera image input received from the camera subsystem includes the representation of the iris from the human eye.

18. The method of claim 11, further comprising storing the diagnostic image and the diagnostic results to memory.

19. The method of claim 18, further comprising collecting user identification information.

20. The method of claim 19, further comprising sending the saved diagnostic image, the saved diagnostic results, and the user identification information to a designated recipient as part of an electronic message.

21. A mobile device comprising:
a memory;
a display subsystem;
a camera subsystem; and
a programmable processor operatively coupled to the memory, the display subsystem, and the camera subsystem and configured to execute non-transitory computer-executable code, the code instructing the programmable processor to:
determine whether a camera image input received from the camera subsystem includes a representation of an iris from a human eye;
display the camera image input using the display subsystem until the candidate image is captured, wherein iris image alignment guidelines include an alignment circle displayed as an overlay on the displayed camera image input;
display a time out message using the display subsystem in response to being unable to determine, after a predetermined period of time, that the camera image input includes the representation of the iris;
capture a candidate image from the camera image input in response to determining that the camera image input includes the representation of the iris, the candidate image having a predetermined number of pixels;
display the candidate image in response to the candidate image being captured;
analyze the representation of the iris in the candidate image, in response to the candidate image being captured, to determine whether the candidate image is suitable for further processing as a diagnostic image based upon the representation of the iris in the candidate image meeting predetermined criteria;
perform a diagnostic analysis on the representation of the iris in the diagnostic image in response to determining that the candidate image is suitable for further processing as the diagnostic image, the diagnostic analysis arriving at diagnostic results; and
display the diagnostic results using the display subsystem in response to the diagnostic analysis being performed.
